(19) United States
(12) Patent Application Publication
(10) Pub. No.: US 2005/0153937 A1
(43) Pub. Date: Jul. 14, 2005

(54) Desensitization of compliment activation using monocyte/macrophage inhibitory compounds

(76) Inventor: Gershon Golomb, Efrat (IL)

Correspondence Address: MORGAN & FINNEGAN, L.L.P., 3 World Financial Center, New York, NY, 10281-2101, US

(21) Appl. No.: 10/982,449

(22) Filed: Nov. 4, 2004

Related U.S. Application Data
(60) Provisional application No. 60/518,405, filed on Nov. 7, 2003.

(51) Int. Cl. A61K 31/663; A61K 31/675

(52) U.S. Cl. 514/89; 514/102

(57) ABSTRACT

The present invention relates to methods and compositions designed for the prevention, reduction, treatment, or management of complement activation-related acute hypersensitivity also known as C-mediated pseudoallergy (CARPA) that results from the administration of a medicinal composition including, but not limited to, those compositions in liposomal, micellar, nanoparticle, emulsion, and colloidal formulations. The methods of the invention comprise the administration of an effective amount of one or more therapeutic agents containing one or more active compounds which specifically decreases or inhibits the activity of and/or eliminates or diminishes the amount of macrophages and/or monocytes. In preferred embodiments, the active compound is a bisphosphonate.

This application claims benefit of U.S. provisional application No. 60/518,405, filed Nov. 7, 2003, which is incorporated herein by reference in its entirety.

### FIELD OF THE INVENTION

The present invention relates to methods and compositions designed for the prevention, reduction, treatment, or management of complement activation-related acute hypersensitivity also known as C-mediated pseudoallergy (CARPA). In preferred embodiments, CARPA associated with medicinal composition administration including, but not limited to, those compositions in liposomal, micellar, nanoparticle, emulsion, and colloidal formulations is prevented, reduced, treated, or managed. The methods of the invention comprise the administration of an effective amount of a one or more therapeutic agents containing an active compound in a formulation which specifically decreases or inhibits the activity of and/or eliminates or diminishes the amount of macrophages and/or monocytes. In some embodiments, the active compound is a bisphosphonate. In more specific embodiments, the bisphosphonate is an alendronate.

### BACKGROUND OF THE INVENTION

Medicinal compositions have transformed man's survival rates in the past decades. However, many pharmaceutical compositions when administered to a mammal cause an adverse reaction in some patients. In some cases, the allergic reaction is severe and can be life threatening. Examples of medicinal compositions recently associated with a strong adverse reaction are those in liposomal, micellar, nanoparticle, emulsion, and colloidal formulations.

The use of liposomal, micellar, nanoparticle, emulsion, or colloidal formulations in medicinal compositions is gaining widespread recognition and use in the medical field. Many drugs are now sold as emulsions, suspensions, solutions with surface active agents to enhance drug solubility and liposomal drug formulations, in the hopes that the drugs will be more effectively delivered to their target organ or cell type. For example, the nonionic emulsifier vehicle “Cremophor EL” is known for its use with an extensive array of pharmaceuticals, including Miconazole (Handbook on Injectable Drugs), echinomycin (Handbook on Injectable Drugs), teniposide (Rowinsky et al. 1992, Seminars in Oncology, 19, 646), diazepam (Lau et al. 1989, Int. J. Pharm. 54, 171) althesin (Dye et al., 1980, Br. Med. J. 230, 1353) and paclitaxel (Rowinsky et al., 1992, supra). However, many of these formulations cause a hypersensitivity reaction in patients which necessitates ceasing treatment with the formulation, thereby depriving the patient of the beneficial actions of the drug. Additionally, medicinal compositions in liposomal formulations such as Doxil and Ambisome or micellar formulations such as Taxol similarly can cause hypersensitivity reactions in patients (Chanan-Khan et al., 2003, Ann Oncol. 14: 1430-7; Cesaro et al., 1999, Support Care Cancer 7: 284-6; Weiss et al., 1990, J. Clin Oncol. 8: 1263-8).

Clinical studies with medicinal compositions that elicit hypersensitivity reactions have revealed that the reaction often develops immediately after the start of infusion and includes symptoms of cardiopulmonary distress such as dyspnea, tachypnea, tachcardia, hypotension and hypertension, chest pain, and back pain. These reactions have been termed complement activation-related acute hypersensitivity or C-mediated pseudoallergy (CARPA). Unlike IgE-mediated (type I) allergy, CARPA usually arises at the first exposure to the drug without prior sensitization, and the symptoms usually lessen or disappear on subsequent treatments (tachyphylaxis). The frequency of such reactions among patients treated with liposomal drugs, such as, for example, Doxil is 6.8%, which is comparable to the incidence rate reported in other liposome drug trials. (Dezube, B. J., 1996 In: Doxil Clinical Series, Califon, N.J. Gardiner-Caldwell SynerMed, p. 1-8). These reactions can be life-threatening in some 0.9% of the patients, thus precluding further treatment with the liposomal formulations. Also, micellar formulations of Taxol elicit minor reactions (such as flushing or rash) in 41%-44% of all patients. However, 1.5%-3% of taxol-treated patients have a much more serious reaction, including anaphylactoid reaction, dyspnea, hypertension and flushing, necessitating discontinuation of the drug and appropriate first aid (Rowinsky et al., 1993, Semin. Oncol. 204: 1-15 and Weiss et al., 1990, J. Clin Oncol. 8: 1263-8). Attempts to prevent Taxol cardiotoxicity and anaphylactoid reaction have included reliance on pretreatment of patients with antihistamine and corticosteriods, and by prolonging the infusion time of the drug. Moreover, complement activation is also associated with intravenous injections/infusions of polymeric nanoparticles.

Thus, it is an object of the present invention to provide a method for preventing hypersensitivity reactions associated with the administration of complement-activating medicinal compositions.

### SUMMARY OF THE INVENTION

The present invention relates to methods and compositions designed for the prevention, reduction, treatment, or management of complement activation-related acute hypersensitivity also known as C-mediated pseudoallergy (CARPA). In preferred embodiments, CARPA associated with administration of a medicinal composition including, but not limited to, those compositions in liposomal, micellar, nanoparticle, emulsion, colloidal formulations is prevented, reduced, treated, or managed. The methods of the invention comprise the administration to a patient in need thereof an effective amount of a one or more therapeutic agents containing an active compound in a formulation which specifically decreases or inhibits the activity of and/or eliminates or diminishes the amount of macrophages and/or monocytes such that complement activation is inhibited or decreased and thus cannot cause a hypersensitivity reaction. Administration of one or more therapeutic agents according to the invention acts as an acute, short term treatment primarily aimed at inhibiting or ameliorating CARPA side effects caused by administration of a medicinal composition. One or more therapeutic agents is administered to a patient prior to, concurrently with, or after the administration of a complement-activating medicinal composition. In some embodiments, the patient administered a composition of the invention has had a prior CARPA episode in response to a prior administration of a medicinal composition. In other embodiments, the patient administered a composition of the invention has not yet had a CARPA episode but is undergoing treatment with a known complement-activating medicinal composition.

In preferred embodiments, the therapeutic agent specifically targets macrophages and/or monocytes. Because macrophages and monocytes are phagocytic cells, in these embodiments, the therapeutic agents are prepared such that they comprise particles of such properties as to enter into a cell primarily or exclusively via phagocytosis. The therapeutic agent comprises an active compound in a formulation such that the physiochemical properties, e.g. size or charge, of the formulation can be internalized only or primarily by phagocytosis. The therapeutic agent may comprise an encapsulated active compound or a particulate active compound. Once phagocytosed, the active compound is released from the formulation into the targeted cell, e.g., macrophages and monocytes, and inhibits the function of and/or destroys the cell. In preferred embodiments, the active compound in the therapeutic agent is a bisphosphonate. In more preferred embodiments, the bisphosphonate is an alendronate.

In one embodiment, the present invention relates to a method of preventing, reducing, treating, or managing CARPA by administering to an individual in need thereof an effective amount of one or more therapeutic agents comprising an encapsulated active compound. In another embodiment, the present invention relates to a method of preventing, reducing, treating, or managing CARPA by administering to an individual in need thereof an effective amount of one or more therapeutic agents comprising a particulate active compound. The active compound is encapsulated in a suitable carrier of a specific dimension or made into particulates of a specific dimension. The therapeutic agent specifically targets macrophages and/or monocytes by virtue of the properties of its formulation, such as, for example, size and/or charge, which allow the therapeutic agent to be taken-up primarily or exclusively by phagocytosis. Once the formulation is taken-up by the cell, the active compound is released from the encapsulating carrier or the particulate and the agent is able to inhibit the activity of and/or destroy the phagocyte.

In a further embodiment, the present invention includes a pharmaceutical composition for administration to subjects in need thereof a therapeutic agent comprising an active compound in a formulation selected from the group consisting of an encapsulated active compound and a particulate active compound together with a pharmaceutically acceptable vehicle, carrier, stabilizer or diluent for the prevention, reduction, treatment, or management of CARPA. Those subjects in need of such administration are those who i) have recently undergone treatment with a complement-activating medicinal composition, ii) are currently undergoing treatment with a complement-activating medicinal composition, or iii) will be undergoing treatment with a complement-activating medicinal composition in the near future.

The therapeutic agent of the present invention comprises an active compound in a formulation that is preferably in the size range of 0.03-1.0 μm. However, depending on the type of agent and/or the carrier used, the more preferred ranges include, but are not limited to, 0.07-0.5 μm, 0.1-0.3 μm and 0.1 to 0.18 μm.

### DETAILED DESCRIPTION OF THE INVENTION

The present invention relates to methods and compositions designed for the prevention, reduction, treatment, or management of complement activation-related acute hypersensitivity also known as C-mediated pseudoallergy (CARPA). Once activated, the complement system causes hypersensitivity reactions with the help of macrophages and monocytes. During these reactions, massive increases in macrophage/monocyte secretion products are observed in the blood of the affected patient. Thus, it is an object of the present invention to prevent, reduce, treat, or manage CARPA by inhibiting or reducing macrophage and/or monocyte activity either directly or indirectly through the inhibition or reduction of activity and/or reduction in numbers of circulating cells. In preferred embodiments, the CARPA is caused by the administration of a complement-activating medicinal compound including, but not limited to, those compositions in liposomal, micellar, nanoparticle, emulsion, and colloidal formulations. Therefore, methods of the invention can be used to decrease or eliminate the severe undesirable side effects associated with administration of complement-activating medicinal compositions in some patients.

The present invention relates to methods and compositions designed to decrease or inhibit the activity of and/or eliminate or diminish the amount of macrophages and/or monocytes for an acute, short term period preceding, during, or following administration of a complement-activating medicinal composition such that the medicinal composition can be tolerated by the patient. The methods of the invention comprise the administration of an effective amount of a formulation containing one or more therapeutic agents which specifically decreases or inhibits the activity of and/or eliminates or diminishes the amount of macrophages and/or monocytes in a patient.

The therapeutic agents used in the methods of the invention specifically decrease or inhibit the activity of macrophages and/or monocytes and/or eliminate or diminish the amount of macrophages and/or monocytes in a patient. Specificity of the therapeutic agents is due to the formulation of the active compound in the therapeutic agent such that the active compound has the ability to affect only particular cell types (e.g., macrophages and/or monocytes). In preferred embodiments, specificity of the formulation for phagocytic cells is due to the physiochemical properties, e.g. size or charge, of the formulation such that it can only or primarily be internalized by phagocytosis. Once phagocytosed and intracellular, the active compound inhibits or decreases the activity of the phagocytic cell and/or destroys the phagocytic cell. Although not intending to be bound by any particular mechanism of action, the therapeutic agents of the active compounds are released upon becoming intracellular before disabling an/or destroying the phagocytic cell.

The formulation of the active compound in the therapeutic agent, e.g., encapsulated or particulate, suppresses the hypersensitivity response caused by complement activation by transiently depleting and/or inactivating cells that are important triggers in the hypersensitivity response, namely macrophages and/or monocytes. The therapeutic agents are taken-up, by way of phagocytosis, by the macrophages and monocytes. In contrast, non-phagocytic cells are incapable of taking up the therapeutic agents due to the large dimension and/or other physiochemical properties of the formulation.

The term “phagocytosis” as used herein refers to a preferred means of entry into a phagocytic cell and is well understood in the art. However, the term should be understood to also encompass other forms of endocytosis which may also accomplish the same effect. In particular, it is understood that receptor-mediated endocytosis and other cellular means for absorbing/internalizing material from outside the cell are also encompassed by the methods and compositions of the present invention.

The present invention also relates to a pharmaceutical composition for prevention, reduction, treatment, or management of a hypersensitivity reaction in a mammal comprising administering to a mammal in need thereof one or more therapeutic agents comprising an encapsulated or particulate active compound and a pharmaceutically acceptable excipient, diluent or carrier. In preferred embodiments, at least one of the active compounds is a bisphosphonate. In more preferred embodiments the bisphosphonate is encapsulated in a liposome.

### Active Compounds

The active compounds used in the therapeutic agents and in the methods of the invention specifically decrease or inhibit the activity of macrophages and/or monocytes and/or eliminate or diminish the amount of macrophages and/or monocytes in a patient, by virtue of the physiochemical properties, such as size or charge, of the formulation. The active compound may be an intracellular inhibitor, deactivator, toxin, arresting substance and/or cytostatic/cytotoxic substance that, once inside a phagocytic cell such as a macrophage or monocyte, inhibits, destroys, arrests, modifies and/or alters the phagocytic cell such that it can no longer function normally and/or survive.

As used herein, the term “active compounds” refers to molecules which are encapsulated or particularized to make up all or part of the therapeutic agent and provide the inactivating/toxic potency to the formulation, e.g., inhibits or decreases macrophage and/or monocyte activity and/or eliminates or decreases the amount of macrophages and/or monocytes. Compounds that can be active compounds include, but are not limited to, inorganic or organic compounds; or a small molecule (less than 500 daltons) or a large molecule, including, but not limited to, inorganic or organic compounds; proteinaceous molecules, including, but not limited to, peptide, polypeptide, protein, post-translationally modified protein, antibodies etc.; or a nucleic acid molecule, including, but not limited to, double-stranded DNA, single-stranded DNA, double-stranded RNA, single-stranded RNA, or triple helix nucleic acid molecules. Active compounds can be natural products derived from any known organism (including, but not limited to, animals, plants, bacteria, fungi, protista, or viruses) or from a library of synthetic molecules. Active compounds can be monomeric as well as polymeric compounds.

In preferred embodiments the active compound is a bisphosphonate or analog thereof. The term “bisphosphonate” as used herein, denotes both geminal and non-geminal bisphosphonates. In a specific embodiment, the bisphosphonate has the following formula (I): 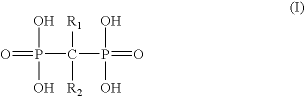
wherein R₁ is H, OH or a halogen atom; and R₂ is halogen; linear or branched C₁-C₁₀ alkyl or C₂-C₁₀ alkenyl optionally substituted by heteroaryl or heterocyclyl C₁-C₁₀ alkylamino or C₃-C₈ cycloalkylamino where the amino may be a primary, secondary or tertiary; —NHY where Y is hydrogen, C₃-C₈ cycloalkyl, aryl or heteroaryl; or R₂ is —SZ where Z is chlorosubstituted phenyl or pyridinyl.

In a more specific embodiment, the bisphosphonate is an alendronate or an analog thereof. Alendronate includes its various salts including, but not limited to, calcium, sodium, and other similar salts and esters. Alendronate also exists in various hydration states and these are also encompassed herein, including, but not limited to, alendronate monohydrates, dihydrates, and trihydrates, as well as anhydous forms of the compound. In such an embodiment, the alendronate has the following formula (II):

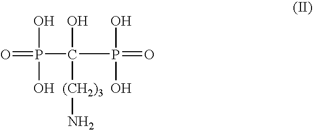

In other specific embodiments, additional bisphosphonates can be used as active compounds in the methods of the invention. Examples of other bisphosphonates include, but are not limited to, clodronate, tiludronate, 3-(N,N-dimethylamino)-1-hydroxypropane-1,1-diphosphonic acid, e.g. dimethyl-APD; 1-hydroxy-ethylidene-1,1-bisphosphonic acid, e.g. etidronate; 1-hydroxy-3(methylpentylamino)-propylidene-bisphosphonic acid, (ibandronic acid), e.g. ibandronate; 6-amino-1-hydroxyhexane-1,1-diphosphonic acid, e.g. amino-hexyl-BP; 3-(N-methyl-N-pentylamino)-1-hydroxypropane-1,I-diphosphonic acid, e.g. methyl-pentyl-APD; 1-hydroxy-2-(imidazol-1-yl)ethane-1,1-diphosphonic acid, e.g. zoledronic acid; 1-hydroxy-2-(3-pyridyl)ethane-I,I-diphosphonic acid (risedronici acid), e.g. risedronate; 3-[N-(2-phenylthioethyl)-N-methylamino]-1-hydroxypropane-1,1-bishosphonic acid; 1-hydroxy-3-(pyrrolidin-1-yl)propane-1,1-bisphosphonic acid, 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U81581 (Upjohn); and 1-hydroxy-2-(imidazo[1,2-a]pyridin-3-yl)ethane-1,1-diphosphonic acid, e.g. YM 529, 2-(2-aminopyrimidinio) ethylidene-1,1-bisphosphonic acid betaine (ISA-13-1), or analogs thereof.

The present invention also encompasses therapeutic agents containing other active compounds including, but not limited to, gallium, gold, selenium, gadolinium, silica, mithramycin, sirolimus, everolimus, and other similar analogs thereof. Generally, chemotherapeutic agents, such as, for example, 5-fluorouracil, cisplatinum, alkylating agents and other anti-proliferation or anti-inflammatory compounds, such as, for example, steroids, aspirin and non-steroidal anti-inflammatory drugs may also be used as active compounds.

The present invention is meant to encompass the administration of one or more therapeutic agents to prevent, reduce, treat, or manage complement activation-related acute hypersensitivity also known as C-mediated pseudoallergy (CARPA). More than one therapeutic agent can be administered in combination to the patient. The therapeutic agents administered in combination may have different active compounds in the same or different formulation (e.g., encapsulated or particulate) or the same active compound in different formulations. The term “in combination” is not limited to the administration of the therapeutic agents at exactly the same time, but rather it is meant that the therapeutic agents are administered to a patient in a sequence and within a time interval such that they can act together to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route which effectively transports the therapeutic agent to the appropriate or desirable site of action.

### Identification of Active Compounds

The invention provides methods of screening for compounds that can be used as an active compound. Although not intending to be bound by a particular mechanism of action, a compound that is an active compound for use in the methods of the invention can, once targeted to the macrophage and/or monocyte by the physiochemical properties of the formulation itself, i) inhibit phagocyte activity, ii) decrease phagocyte activity, iii) eliminate macrophages/monocytes from circulation, and/or iv) decrease the number of macrophages and/or monocytes in circulation.

The methods of screening for active compounds generally involve incubating a candidate therapeutic agent with phagocytic cells (e.g., macrophages and/or monocytes) either in vitro or in vivo and then assaying for an alteration (e.g., decrease) in phagocytic cell activity or longevity thereby identifying an active compound for use in the present invention. Any method known in the art can be used to assay phagocytic cell activity or longevity.

In one embodiment, phagocytic activity is assayed by the level of cell activation in response to an activating stimulus. For example, macrophage/monocyte activation can be assayed by quantifying the levels of chemotactic factors such as macrophage chemoattractant protein-1 (MCP-1) macrophage inflammatory protein-1 alpha (MIP-1 alpha) as well as other substances produced by macrophages such as interleukin 1 beta (IL-1β), tissue necrosis factor alpha (TNF-α), histamine, tryptase, PAF, and eicosanoids such as TXA₂, TXB₂, LTB₂, LTB₄, LTC₄, LTD₄, LTE₄, PGD₂ and TXD₄. Any methods known in the art can be used to assay levels of phagocytic secretion products including, but not limited to, ELISA, immunoprecipitation, and quantitative western blot.

In another embodiment, phagocyte longevity is assayed. For example, cell proliferation can be assayed by measuring ³H-thymidine incorporation, by direct cell count, by detecting changes in transcriptional activity of known genes such as proto-oncogenes (e.g., fos, myc) or cell cycle markers; or by trypan blue staining. Any method known in the art can be used to assay for levels of mRNA transcripts (e.g., by northern blots, RT-PCR, Q-PCR, etc.) or protein levels (e.g., ELISA, western blots, etc.).

In one embodiment, an agent that decreases the activity of phagocytes is identified by:
a) contacting a phagocyte with a first agent and a second agent, said first agent being an agent which activates said phagocyte and said second agent being a candidate agent; and
b) determining the level of activation in said contacted phagocyte,
   wherein a decrease in activation in said contacted phagocyte as compared to the level of activation in a phagocyte contacted with said first agent in the absence of said second agent (i.e., a control cell) indicates that said second agent decreases the activity of a phagocyte.

In another embodiment, an agent that decreases the amount of phagocytes is identified by:
a) contacting a phagocyte with an agent; and
b) determining the viability of said contacted phagocyte,
   wherein a decrease in viability in said contacted phagocyte as compared to the viability of a phagocyte not contacted with said agent (i.e., a control cell) indicates that said agent decreases phagocytes.

In other embodiments, candidate agents are assayed for their ability to alter phagocyte activity or longevity in a manner that is substantially similar to or better than agents known to alter phagocyte activity or longevity in a therapeutically desirable way. As used herein “substantially similar to” refers to an agent having similar action on a phagocyte as an exemplified agent, i.e., an agent that inhibits the activity, function, motility, and/or depletion of phagocytes.

Additionally, candidate agents may be used in animal models of CARPA to assess their ability to be used in the methods of the invention. Animal models may be used as a first assay to determine if a candidate agent may be used as an active agent as well as a second assay to confirm the utility of agents found to have desirable activity in in vitro assays.

In one embodiment, the animal model used is a pig CARPA model (see e.g., Section 6). Intravenous injections of small amounts of certain liposomes in pigs leads instantly to significant hemodynamic and cardiopulmonary changes and skin alterations typical of anaphylactic or anaphylactoid shock. The reaction is associated with massive rises of macrophage secretion products (especially TXA₂) in the blood. These reactions can be lethal without resuscitation with epinephrine, cardiac massage, and/or electroshock. This reaction in pigs closely resembles the hypersensitivity syndrome observed in a relatively high proportion (2-45%) of humans after infusion of certain liposomal and anticancer drugs. For example, the bolus dose of Doxil that causes cardiopulmonary distress in pigs corresponds to the dose that reaches the human blood during the first 20-60 seconds of infusion and causes CARPA in sensitive individuals. Unlike in humans, liposome-induced CARPA in pigs shows relatively small biological variation (e.g., essentially all pigs react similarly to a certain reactogenic liposome preparation) thus the model is highly reproducible.

### Formulations of Active Compounds

Therapeutic agents comprise active compounds in formulations such that the active compound is in particles that are large enough to only or primarily be internalized by phagocytosis, thus imparting specificity to macrophages and monocytes. Although non-phagocytic cells may be affected by the active compound should it become intracellular, there is no mechanism for a non-phagocytic cell to internalize the active compound when formulated in this manner (i.e., as a therapeutic agent). Therapeutic agents comprise active compounds formulated preferably in the size range of 0.03-1.0 μm, more preferably 0.07-0.5 μm, more preferably 0.1-0.3 μm, and more preferably 0.1-0.18 μm. However, this is merely an example and other size ranges may be used without departing from the spirit or scope of the invention.

Any method known in the art can be used to incorporate an active compound into a formulation such that it can only or primarily be internalized via phagocytosis. Formulations of active compounds (i.e., therapeutic agents) may sequester the active compound for a sufficient time to enhance delivery of the compound to the target site. Furthermore, formulations of active compounds may discharge the compound from the particles when they are within the target cell (e.g., the macrophage or monocyte) at the target site. Thus, only bisphosphonates in an insoluble form (e.g., encapsulated or particulate) are present when the therapeutic agent is extracellular.

In one embodiment, the active compound is encapsulated in a carrier (i.e., encapsulating agent) of desired properties. The term “encapsulated active compound” includes an active compound which is encapsulated, embedded, and/or adsorbed within particle, dispersed in the particle matrix, adsorbed or linked on a surface of the particle, or a combination of any of these forms. The particles include, but are not limited to, inert polymeric particles, such as microcapsules, nanocapsules, nanospheres, microspheres, nanoparticles, microparticles, and liposomes.

In a specific embodiment, the encapsulating agent is a liposome. The liposomes may be prepared by any of the methods known in the art (see, e.g., Mönkkönen, J. et al., 1994, J. Drug Target, 2: 299-308; Mönkkönen, J. et al., 1993, Calcif. Tissue Int., 53: 139-145; Lasic D D., Liposomes Technology Inc., Elsevier, 1993, 63-105 (chapter 3); Winterhalter M, Lasic D D, Chem Phys Lipids, 1993 September; 64(1-3): 35-43). The liposomes may be positively charged, neutral or, more preferably, negatively charged. The liposomes may be a single lipid layer or may be multilamellar. Suitable liposomes in accordance with the invention are preferably non-toxic liposomes such as, for example, those prepared from phosphatidyl-choline phosphoglycerol and cholesterol. The diameter of the liposomes used preferably ranges from 0.03-1.0 μm, and more preferably 100-300 nm. However, other size ranges suitable for phagocytosis by macrophages and/or monocytes may also be used.

In another specific embodiment, the encapsulating agent is an embedding agent such that the active compound is embedded in a carrier of desired properties. An active compound which is encapsulated by embedding includes those active compounds that are embedded, enclosed, and/or adsorbed within a carrier, dispersed in the carrier matrix, adsorbed or linked on the carrier surface, or a combination of any of these forms. In specific embodiments, the embedding agent (or carrier) is a microparticle, nanoparticle, nanosphere, microsphere, microcapsule, or nanocapsule (see e.g., M. Donbrow in: Microencapsulation and Nanoparticles in Medicine and Pharmacy, CRC Press, Boca Raton, Fla., 347, 1991). The term carrier includes both polymeric and non-polymeric preparations. In a more specific embodiment, the encapsulating agent is a nanoparticle. Preferably, nanoparticles are 0.03-1.0 μm in diameter and can be spherical, non-spherical, or polymeric particles. The active compound may be embedded in the nanoparticle, dispersed uniformly or non-uniformly in the polymer matrix, adsorbed on the surface, or in combination of any of these forms. In a preferred embodiment, the polymer used for fabricating nanoparticles is biocompatible and biodegradable, such as poly(DL-lactide-co-glycolide) polymer (PLGA). However, additional polymers which may be used for fabricating the nanoparticles include, but are not limited to, PLA (polylactic acid), and their copolymers, polyanhydrides, polyalkyl-cyanoacrylates (such as polyisobutylcyanoacrylate), polyethyleneglycols, polyethyleneoxides and their derivatives, chitosan, albumin, gelatin and the like.

In another embodiment, the therapeutic agent is in particulate form, the particles each being of desired properties. A particulate active compound includes any insoluble suspended or dispersed particulate form of the active compound which is not encapsulated, entrapped or absorbed within a carrier. An active compound which is in particulate form includes those active compounds that are suspended or dispersed colloids, aggregates, flocculates, insoluble salts, insoluble complexes, and polymeric chains of an agent. Such particulates are insoluble in the fluid in which they are stored/administered (e.g., saline or water) as well as the fluid in which they provide their therapeutic effect (e.g., blood or serum). Typically, “insoluble” refers to a solubility of one (1) part of a particulate active compound in more than ten-thousand (10,000) parts of a solvent. Any method known in the art to make particulates or aggregates can be used. Preferably, particulates are 0.03-1.0 μm in diameter and can be any particular shape.

### Determination of Particle Size

Therapeutic agents comprise active compounds that are preferably formulated such that the size of the active compound particle (e.g., encapsulated or particularized active compound) is large enough to only or primarily be internalized by phagocytosis, that is, preferably larger than 0.03 μm. In preferred embodiments, such formulations are 0.03-1.0 μm, more preferably 0.07-0.51 μm, more preferably 0.1-0.3 μm, and most preferably 0.1 to 0.18 μm. Any method known in the art can be used to determine the size of the particles in the therapeutic agent before administration to a patient in need thereof. For example, a Nicomp Submicron Particle Sizer (model 370, Nicomp, Santa Barbara, Calif.) or a Malvern Zetasizer Nano ZS (model ZS-ZEN3600, Malvern, Worcestershire, United Kingdom) utilizing laser light scattering can be used.

Methods can be used to encapsulate or particularize active compounds that produce particles of varying sizes, including those smaller than in the preferred embodiments. Any method known in the art may be used to separate the encapsulated or particularized active compounds that are of the desired size from those that are outside the range (e.g., too small or too large) of the desired size. Therapeutic agents may include only or primarily those particles of active compound that have been determined to be within a desired size range.

### Complement-Activating Medicinal Compositions

In preferred embodiments, methods and compositions of the present invention are used to prevent, decrease, treat, or manage CARPA caused by administration of any complement-activating medicinal composition. A medicinal composition is complement-activating if administration of a therapeutically effective amount of the composition causes the activation of complement via the classical pathway, the alternative pathway, or any combination of each. As a result of complement activation by the medicinal composition, the patient experiences a hypersensitivity reaction (e.g., CARPA).

In one embodiment, a medicinal composition is complement activating if, after administration of said medicinal composition to a patient, the patient experiences one or more of the following symptoms: cardiopulmonary distress (e.g., dyspnea, tachypnea, tachcardia, chest pain, back pain, etc.), alteration in blood pressure, increased pulmonary arterial pressure, alteration in systemic arterial pressure, increased heart rate, decreased cardiac output, decreased PCO₂ in exhaled air, decreased capillary PO₂, increased pulmonary and systemic vascular resistance, flare or flushing of the skin, and EKG alterations (e.g., tachycardia, bradycardia, arrhythmia, ST-depression, T-wave inversion) or the like. In another embodiment, a medicinal composition is complement activating if, after administration of said medicinal composition to a patient, the patient has an increased amount of macrophage secretion products (e.g., histamine, tryptase, PAF, and eicosanoids such as TXA₂, TXB₂, LTB₂, LTB₄, LTC₄, LTD₄, LTE₄, PGD₂ and TXD₄) in the blood as compared to the levels of secretion products prior to administration.

In one embodiment, the complement-activating medicinal composition exhibits tachyphylaxis (e.g., a decrease in symptoms upon added exposure to the medicinal composition). In another embodiment, the complement-activating medicinal composition does not exhibit tachyphylaxis. In other embodiments, the complement-activating medicinal composition is in a liposomal, micellar, nanoparticle, emulsion, or colloidal formulation.

Examples of complement-activating medicinal compositions include, but are not limited to, Doxil (and other complement-activating formulations of doxorubicin), Ambisome and Abelcet (and other complement-activating formulations of amphotericin B), DaunoXome (and other complement-activating formulations of daunorubicin), Neoral and Sandimmune (and other complement-activating formulations of cyclosporine), Vumon (and other complement-activating formulations of teniposide), Amphocil (and other complement-activating formulations of amphotericin B), Althesin (and other complement-activating formulations of alphaxalone), T-Quil, Valrelease, and Stesolid MR (and other complement-activating formulations of diazepam), Epontol (and other complement-activating formulations of propanidid), Taxol (and other complement-activating formulations of paclitaxel), Taxotere (and other complement-activating formulations of docetaxel), miconazole, and echinomycin.

### Administration of the Formulation

Effective amounts of the therapeutic agents are administered to patients in need thereof for a time period of a single treatment that produces inhibition/depletion of macrophages and/or monocytes. The effect of a treatment on macrophages and/or monocytes preferably lasts for a period that is less than a month, preferably less than two weeks, more preferably less than one week. In some embodiments, therapeutic agents are administered as a short term, acute therapy. In other embodiments, therapeutic agents are administered as a chronic therapy. Empirically, one can determine this by administering the compound to an individual in need thereof (or an animal model of such an individual) and monitoring the level of inhibition/depletion at different time points. One may also correlate the time of inhibition with the appropriate desired clinical effect, e.g. reduction in CARPA after administration of a complement-activating medicinal composition.

In one embodiment, 0.01-10 mg/kg of liposomes containing a bisphosphonate, such as an alendronate, is administered to a patient as a CARPA inhibitor. In a more specific embodiment, 0.3-4 mg/kg of liposomes containing a bisphosphonate, such as an alendronate, is administered to a patient as a CARPA inhibitor. In a more specific embodiment, 0.5-2 mg/kg of liposomes containing a bisphosphonate, such as an alendronate, is administered to a patient as a CARPA inhibitor.

In another embodiment, 0.001-1 mg/kg of liposomes containing a bisphosphonate, such as a clondronate, is administered to a patient as a CARPA inhibitor. In a more specific embodiment, 0.03-0.4 mg/kg of liposomes containing a bisphosphonate, such as an clondronate, is administered to a patient as a CARPA inhibitor. In a more specific embodiment, 0.05-0.2 mg/kg of liposomes containing a bisphosphonate, such as an clondronate, is administered to a patient as a CARPA inhibitor.

In other embodiments using other bisphosphonates, the amount of therapeutic agent to be administered for treatment can be determined empirically by one skilled in the art using, e.g., the types of in vitro and in vivo assays described for identification of active compounds supra. Activity of therapeutic agents can be compared where one therapeutic agent comprises a bisphosphonate with a known administration amount and one comprises a bisphosphonate with an unknown administration amount. In this way, one can approximate how the activity level of the bisphosphonate with the unknown administration amount compares to the activity level of the bisphosphonate with known administration amount and thus adjust the amount to be administered accordingly. For example, using the assays, one can determine the amount of liposomes containing pamidronate one must use to approximate the level of activity in the assay of liposomes containing alendronate and thus an indication of the amount of pamidronate containing liposomes that would be the equivalent of 0.01-10 mg/kg of alendronate containing liposomes.

Therapeutic agents of the invention (or pharmaceutical compositions comprising therapeutic agents of the invention) can be administered to a patient prior to, concurrently with, or after the administration of a complement-activating medicinal composition. In one embodiment, a therapeutic agent or pharmaceutical composition thereof is administered to a patient before the administration of a complement-activating medicinal composition. It may be preferred to administer the therapeutic agent or pharmaceutical composition thereof up to 3 days before, 1-6 hours before, within 1 hour before, less than 1 hour before, or within minutes before the administration of the complement-activating medicinal composition. In one embodiment, the patient administered a therapeutic agent or pharmaceutical composition thereof has had a prior CARPA episode in response to a prior administration of a medicinal composition. In another embodiment, the patient administered a therapeutic agent or pharmaceutical composition thereof has not yet had a CARPA episode but is undergoing treatment with a known complement-activating medicinal composition. In yet another embodiment, the patient administered a therapeutic agent or pharmaceutical composition thereof is at risk for developing CARPA (either due to administration of a complement-activating medicinal composition or otherwise).

In another embodiment, a therapeutic agent or pharmaceutical composition thereof is administered to a patient at the same time or substantially the same time as the administration of a complement-activating medicinal composition. In such embodiments, the patient has had a prior CARPA episode in response to a prior administration of a medicinal composition and/or the patient is undergoing treatment with a known complement-activating medicinal composition and/or the patient is at risk for developing CARPA. In a specific embodiment, the therapeutic agent or pharmaceutical composition thereof and the medicinal composition are administered within 10 minutes of each other. In another specific embodiment, the therapeutic agent or pharmaceutical composition thereof and the medicinal composition that is liposomal are mixed together and administered as one composition. In another specific embodiment, the therapeutic agent or pharmaceutical composition thereof and the medicinal composition are mixed together and encapsulated in the same liposomes when liposomal formulations of medicinal compositions are administered.

In another embodiment, a therapeutic agent or pharmaceutical composition thereof is administered to a patient exhibiting symptoms of CARPA including, but not limited to cardiopulmonary distress (e.g., dyspnea, tachypnea, tachcardia, chest pain, back pain, etc.), alteration in blood pressure, increased pulmonary arterial pressure, alteration in systemic arterial pressure, increased heart rate, decreased cardiac output, decreased PCO₂ in exhaled air, decreased capillary PO₂, increased pulmonary and systemic vascular resistance, flare or flushing of the skin, EKG alterations, and/or increased one or more macrophage secretion products (e.g., histamine, tryptase, PAF, and eicosanoids such as TXA₂, TXB₂, LTB₂, LTB₄, LTC₄, LTD₄, LTE₄, PGD₂ and TXD₄) in the blood. Other symptoms will be apparent to the skilled artisan and medical doctor.

In embodiments where the therapeutic agent or pharmaceutical composition thereof and the complement-activating medicinal composition are administered apart, said administrations can occur less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In a specific embodiment, the therapeutic agent or pharmaceutical composition thereof and the complement-activating medicinal composition are administered within the same patient visit. In another specific embodiment, the therapeutic agent or pharmaceutical composition thereof is administered first. In another specific embodiment, the complement-activating medicinal composition is administered first.

The skilled person can readily determine the appropriate dose and timing of administration depending on various physiological factors specific to the individual patient (such as, for example, weight, medical history and genetic predisposition), various factors which influence the anticipated risk of CARPA (such as the type of medicinal composition administered), and the type of formulation being used (e.g., encapsulated, embedded, particulate, etc.).

### Characterization of Therapeutic Utility

The term “effective amount” denotes an amount of a particular therapeutic agent which is effective in achieving the desired therapeutic result, namely inhibited or decreased macrophage and/or monocyte activity and/or elimination or reduction in the amount of macrophages and/or monocytes. In one embodiment, the desired therapeutic result of inhibiting or decreasing macrophage and/or monocyte activity and/or eliminating or reducing in the amount of macrophages and/or monocytes inhibits complement activation. In another embodiment, the desired therapeutic result of inhibiting or decreasing macrophage and/or monocyte activity and/or eliminating or reducing in the amount of macrophages and/or monocytes inhibits the increased complement activation that results from the administration of a complement-activating medicinal compound. In another embodiment, the desired therapeutic result of inhibiting or decreasing macrophage and/or monocyte activity and/or eliminating or reducing in the amount of macrophages and/or monocytes inhibits or lessens CARPA that results from the administration of a complement-activating medicinal compound.

Toxicity and efficacy of the therapeutic methods of the instant invention can be determined by standard pharmaceutical procedures in cell cultures or experimental animals, e.g., for determining the LD₅₀ (the dose lethal to 50% of the population), the No Observable Adverse Effect Level (NOAEL) and the ED₅₀ (the dose therapeutically effective in 50% of the population). The dose ratio between toxic and therapeutic effects is the therapeutic index and it can be expressed as the ratio LD₅₀/ED₅₀ or NOAEL/ED₅₀. Formulations that exhibit large therapeutic indices are preferred. While formulations that exhibit toxic side effects may be used, care should be taken to design a delivery system that targets the agents of such formulations to the site of affected tissue in order to minimize potential damage to unaffected cells and, thereby, reduce side effects.

The data obtained from the cell culture assays and animal studies can be used in determining a range of dosage of the formulation for use in humans. The dosage of such formulations lies preferably within a range of circulating concentrations that include the ED₅₀ with little or no toxicity. The dosage may vary within this range depending upon the dosage form employed and the route of administration utilized. For any formulation used in the method of the invention, the effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The protocols and compositions of the invention are preferably tested in vitro, and then in vivo, for the desired therapeutic activity, prior to use in humans. One example, of such an in vitro assay is an in vitro cell culture assay in which phagocytes (e.g., macrophages and/or monocytes) are grown in culture, and exposed to or otherwise administered a therapeutic agent, and observed for an effect of this assay upon the cells, e.g., inhibited or decreased activity and/or complete or partial cell death. The phagocytic cells may be obtained from an established cell line or recently isolated from an individual as a primary cell line. Many assays standard in the art can be used to measure the activity of the formulation on the phagocytic cells; for example, macrophage/monocyte activation can be assayed by quantitating the levels of chemotactic factors such as macrophage chemoattractant protein-1 (MCP-1), interleukin 1 beta (IL-1β), tissue necrosis factor alpha (TNF-α) and macrophage inflammatory protein-1 alpha (MIP-1 alpha). Many assays standard in the art can be used to assess survival and/or growth of the phagocytic cells; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining.

Selection of the preferred effective dose can be determined (e.g., via clinical trials) by a skilled artisan based upon the consideration of several factors known to one of ordinary skill in the art. Such factors include the severity and type of hypersensitivity reaction, type of complement-activation medicinal composition administered, the therapeutic regime (e.g. whether the therapeutic agent is administered once daily as a slow infusion, a single bolus, several times a day or once every few days), age, body weight, medical history of the patient involved, and other clinical factors influencing the activation of complement such as asthma and other immune or autoimmune conditions, whether the patient is immuno-compromised prior to treatment and other factors known to the skilled artisan to reflect the accuracy of administered pharmaceutical compositions.

### Pharmaceutical Compositions and Routes of Administration

The therapeutic agents used in accordance with the invention may be formulated into pharmaceutical compositions by any of the conventional techniques known in the art (see for example, Alfonso, G. et al., 1995, in: The Science and Practice of Pharmacy, Mack Publishing, Easton Pa., 19th ed.). Formulations comprising one or more therapeutic agents for use in the methods of the invention may be in numerous forms, depending on the various factors specific for each patient (e.g., the severity and type of hypersensitivity reaction, type of complement-activation medicinal composition administered, age, body weight, response, and the past medical history of the patient), the number and type of active compound in the therapeutic agent, the type of formulation (e.g., encapsulated, particulate, etc.), the form of the composition (e.g., in liquid, semi-liquid or solid form), the therapeutic regime (e.g. whether the therapeutic agent is administered once daily as a slow infusion, a single bolus, several times a day or once every few days), and/or the route of administration (e.g., oral, intravenous, intramuscular, intra-arterial, intramedullary, intrathecal, intraventricular, transdermal, subcutaneous, intraperitoneal, intranasal, enteral, topical, sublingual, vaginal, or rectal means). Pharmaceutical carriers, vehicles, excipients, or diluents may be included in the compositions of the invention including, but not limited to, water, saline solutions, buffered saline solutions, oils (e.g., petroleum, animal, vegetable or synthetic oils), starch, glucose, lactose, sucrose, gelatin, malt, rice, flour, chalk, silica gel, sodium stearate, glycerol monostearate, talc, sodium chloride, dried skim milk, glycerol, propylene, glycol, ethanol, dextrose and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms.

Pharmaceutical compositions can be administered systemically or locally, e.g., near the site of injection of the complement-activating medicinal composition. Additionally, systemic administration is meant to encompass administration that can target to a particular area or tissue type of interest.

In certain embodiments, it may be desirable to administer the pharmaceutical of the present invention as a coating on a medical device. Such coating may allow the pharmaceutical composition to be directly applied to a particular site on the patient.

The contents of all published articles, books, reference manuals and abstracts cited herein, are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Modifications and variations of the present invention are possible in light of the above teachings.

### 6. EXAMPLES

The following examples as set forth herein are meant to illustrate and exemplify the various aspects of carrying out the present invention and are not intended to limit the invention in any way.

### 6.1 Surgical Procedures

Adolescent (2040 kg) Yorkshire pigs of both genders are sedated with i.m. ketamine (Ketalar) and then anesthetized with halothane or isoflurane via nose cone. The trachea is intubated to allow mechanical ventilation with an anesthesia machine, using 1-2.5% halothane or isoflurane. A pulmonary artery catheter equipped with thermodilution-based continuous cardiac output detector is advanced via the right internal jugular vein through the right atrium into the pulmonary artery to measure pulmonary arterial pressure (PAP) and cardiac output (CO). Another catheter is inserted into the right femoral artery and advanced into the proximal aorta for blood sampling and to measure systemic arterial pressure (SAP). Blood pressure values and 2-3 leads of the electrocardiogram (ECG) are obtained continually.

### 6.2 Carpa-Activating Compound Injections

The CARPA-activating compounds used in the following experiments are i) Doxil containing 2 mg/ml doxorubicin HCl and 16 mg/ml lipid (phospholipid concentration: 13.3 mM, 150 μg doxorubicin/μmol phospholipid), ii) multilamellar vesicles (MLV) consisting of dimyristoyl phosphatidylcholine (DMPC), dimyristoyl phosphatidylglycerol (DMPG), and cholesterol at a 45:5:50 ratio, and iii) zymosan. Reactogenic doses of Doxil, MLV and zymosan are diluted to 1 ml total volume with PBS or normal saline and injected using 1 ml tuberculin syringes either into the jugular vein (via the introduction sheet) or the pulmonary artery (via the pulmonary catheter). Injections are performed relatively fast (within 10-20 sec) and are followed by 10 ml PBS or saline injections to wash in any vesicles remaining in the void space of the catheter.

### 6.3 Experimental Groups and Treatment

Animals are assigned to an experimental group and treated according to Table 1. Administration of all treatments is by i.v. injection with 30 min intervals between each injection. For all experimental groups, the activator treatment is a series of three consecutive injections consisting of i) Doxil (0.01 mg doxorubicin/kg or 0.08 mg lipid/kg), ii) MLV at 0.25 mg lipid/kg, iii) zymosan at 0.25 mg/kg administered at 30 minute intervals. All animals are sacrificed 30 min after the last injection of the treatment.

TABLE 1

| Group | Treatment | Number |
|---|---|---|
| Vehicle Control | Activator | 2 males |
| | Vehicle | 2 females |
| | Activator | |
| Liposome Control | Activator | 2 males |
| | Empty Liposomes | 2 females |
| | Activator | |
| Desensitization | Activator | 2 males |
| | Liposomes containing | 2 females |
| | Alendronate | |
| | Activator | |
| Pre-desensitization | Liposomes containing | 2 males |
| | Alendronate | 2 females |
| | Activator | |
| | Activator | |

The Vehicle Control group animals are given an injection of Doxil at 0 min, MLV at 30 min, and Zymosan at 60 min as the CARPA-activator treatment. Although each of these injections induces significant (yet non-lethal) cardiopulmonary reactions, all physiologic and blood measures return to baseline within about 30 min after each injection. As a control, the vehicle in the alendronate-containing liposome composition is injected alone (without liposomes) at 90 min. The CARPA-activator is then administered again by injection of Doxil at 120 min, MLV at 150 min, and Zymosan at 180 min. The second series of CARPA-activator injections induce reactions that are substantially identical to those reactions obtained with the first administration of activator (e.g., MLV, Zymosan) or slightly less than those reactions obtained with the first administration of activator yet still significant (e.g., Doxil) thus demonstrating that the vehicle does not show a protective effect alone.

The Liposome Control group animals are treated just as those in the Vehicle Control group except that empty liposomes are injected at 90 min instead of the vehicle. The second series of CARPA-activator injections induce reactions that are substantially identical to those reactions obtained with the first administration of CARPA-activator (e.g., MLV, Zymosan) or slightly less than those reactions obtained with the first administration of activator yet still significant (e.g., Doxil) thus demonstrating that the empty liposomes do not show a protective effect.

The Desensitization group animals are treated just as those in the Vehicle Control group except that liposomes containing alendronate are injected at 90 min instead of the vehicle. The dose of alendronate administered corresponds to approximately 0.3-0.7 mg/kg in humans or the maximal tolerable dose in humans established for other applications. The second series of CARPA-activator injections either do not induce reactions or induce reactions that are significantly decreased as compared to those reactions obtained with the first administration of activator thus demonstrating that the liposomes containing alendronate do show a protective effect.

The Pre-desensitization group animals are given liposomes containing alendronate at 0 min. The dose of alendronate administered corresponds to the maximal tolerable dose in humans established for other applications. The animals are then given two consecutive treatments of activator (e.g., Doxil at 30 min, MLV at 60 min, and Zymosan at 90 min and Doxil at 120 min, MLV at 150 min, and Zymosan at 180 min). Both series of activator injections either do not induce reactions or induce reactions that are significantly decreased as compared to those reactions obtained without prior administration of liposomes containing alendronate thus demonstrating that the liposomes containing alendronate do show a protective effect.

### 6.4 Cardiovascular Variables Measured

Several physiological parameters are measured in the experimental animals before, during, and after treatment (see Section 6.3). Table 2 lists the hemodynamic and biochemical markers that are assayed. Also shown are the changes that are observed in each marker during CARPA (e.g., upon injection of activator) as compared to baseline.

TABLE 2

| | Abnormality observed during |
|---|---|
| Parameter Measured | CARPA |
| Pulmonary arterial pressure (PAP) | Rise |
| Systemic arterial pressure (SAP) | Rise or fall (often biphasic) |
| Cardiac output (CO) | Fall |
| EKG | Tachycardia or bradycardia, |
| | arrhythmia, ST-depression, T-wave |
| | inversion |
| PCO₂ in exhaled air | Fall |
| Capillary PO₂ | Fall |
| Pulmonary and systemic vascular | Rise |
| resistance | |
| Skin color | Flare, flushing (lasting for |
| | 10-20 min) |
| Blood thromboxane A₂ (TXA₂) levels | Rise |
| Blood Histamine levels | Rise or Fall |

1. A method for treating or inhibiting a hypersensitivity reaction comprising administering to a patient in need thereof an effective amount of a therapeutic agent wherein said therapeutic agent comprises one or more encapsulated or particulate active compounds wherein said therapeutic agent is a size that is within a range of 30 nm to 1 μm.

2. The method of claim 1 wherein said hypersensitivity reaction is complement activation-related pseudoallergy (CARPA).

3. The method of claim 1 wherein the therapeutic agent is provided as a single dose.

4. The method of claim 1 wherein the therapeutic agent is provided in multiple doses.

5. The method of claim 1 wherein the therapeutic agent is administered intravenously, intramuscularly, intra-arterially, subcutaneously, or orally.

6. The method of claim 1 wherein the therapeutic agent is applied directly to an affected site on the patient.

7. The method of claim 1 wherein at least one of the one or more active compounds is a bisphosphonate.

8. The method of claim 7 wherein the bisphosphonate is selected from the group consisting of alendronate, pamidronate, clondronate, etidronate, and tiludronate.

9. The method of claim 8 wherein the bisphosphonate is alendronate.

10. The method of claim 1 wherein the therapeutic agent is a liposomal bisphosphonate.

11. The method of claim 10 wherein the bisphosphonate is alendronate.

12. A method of treating a patient in need thereof with a complement-activating medicinal composition without eliciting a hypersensitivity reaction comprising administering i) an effective amount of a therapeutic agent wherein said therapeutic agent comprises one or more encapsulated or particulate active compounds wherein said therapeutic agent is a size that is within a range of 30 nm to 1 μm and ii) said complement-activating medicinal composition.

13. The method of claim 12 wherein the therapeutic agent is provided as a single dose.

14. The method of claim 12 wherein the therapeutic agent is provided in multiple doses.

15. The method of claim 12 wherein the therapeutic agent is administered intravenously, intramuscularly, intra-arterially, subcutaneously, or orally.

16. The method of claim 12 wherein the therapeutic agent is applied directly to an affected site on the patient.

17. The method of claim 16 wherein the therapeutic agent is coated on a medical device.

18. The method of claim 17 wherein the medical device is a stent.

19. The method of claim 12 wherein the therapeutic agent is administered as an infusion that is completed within one hour.

20. The method of claim 12 wherein the therapeutic agent is administered prior to the administration of the complement-activating medicinal composition.

21. The method of claim 20 wherein the therapeutic agent is administered 2 to 3 days before the administration of the complement-activating medicinal composition.

22. The method of claim 12 wherein the therapeutic agent is administered simultaneously with the administration of the complement-activating medicinal composition.

23. The method of claim 12 wherein at least one of the one or more active compounds is a bisphosphonate.

24. The method of claim 23 wherein the bisphosphonate is selected from the group consisting of alendronate, pamidronate, clondronate, etidronate, and tiludronate.

25. The method of claim 24 wherein the bisphosphonate is alendronate.

26. The method of claim 12 wherein the therapeutic agent is a liposomal bisphosphonate.

27. The method of claim 26 wherein the bisphosphonate is alendronate.

28. The method of claim 12 wherein the complement-activating medicinal composition comprises paclitaxel or a pharmaceutical derivative thereof.

29. The method of claim 12 wherein the complement-activating medicinal composition is a liposomal formulation.

30. The method of claim 12 wherein the compliment-activating medicinal composition comprises Doxil.

31. A pharmaceutical composition comprising i) an active compound selected from the group consisting of bisphosphonate, gold, gallium, gadolinium, and selenium; ii) an encapsulating agent; and iii) a pharmaceutically acceptable excipient.

32. The composition of claim 31 wherein the bisphosphonate comprises a geminal bisphosphonate.

33. The composition of claim 31 wherein the bisphosphonate is selected from the group consisting of alendronate, pamidronate, clondronate, etidronate, and tiludronate.

34. The composition of claim 33 wherein the bisphosphonate is alendronate.

35. The composition of claim 31 wherein the encapsulating agent is a liposome.

36. The composition of claim 31 wherein the active compound is encapsulated by the encapsulating agent and is a size that is within a range of 30 nm to 1 μm.

37. A pharmaceutical composition comprising i) a particulate active compound selected from the group consisting of bisphosphonate, gold, gallium, gadolinium, and selenium and ii) a pharmaceutically acceptable excipient.

38. The composition of claim 37 wherein the bisphosphonate comprises a geminal bisphosphonate.

39. The composition of claim 37 wherein the bisphosphonate is selected from the group consisting of alendronate, pamidronate, clondronate, etidronate, and tiludronate.

40. The composition of claim 39 wherein the bisphosphonate is alendronate.

41. The composition of claim 37 wherein the particulate active compound is a size that is within a range of 30 nm to 1 μm.

42. A method for treating or inhibiting a hypersensitivity reaction comprising administering to a patient in need thereof an effective amount of a therapeutic agent wherein said therapeutic agent comprises one or more encapsulated or particulate active compounds wherein said encapsulated or particulate active compounds i) decreases or inhibits the activity of macrophages or monocytes in said patient or ii) diminishes the amount of or eliminates macrophages or monocytes in said patient.

43. The method of claim 42 wherein said hypersensitivity reaction is complement activation-related pseudoallergy (CARPA).

44. The method of claim 42 wherein the therapeutic agent is provided as a single dose.

45. The method of claim 42 wherein the therapeutic agent is provided in multiple doses.

46. The method of claim 42 wherein the therapeutic agent is administered intravenously, intramuscularly, intra-arterially, subcutaneously, or orally.

47. The method of claim 42 wherein the therapeutic agent is applied directly to an affected site on the patient.

48. The method of claim 42 wherein at least one of the one or more active compounds is a bisphosphonate.

49. The method of claim 48 wherein the bisphosphonate is selected from the group consisting of alendronate, pamidronate, clondronate, etidronate, and tiludronate.

50. The method of claim 49 wherein the bisphosphonate is alendronate.

51. The method of claim 42 wherein the therapeutic agent is a liposomal bisphosphonate.

52. The method of claim 51 wherein the bisphosphonate is alendronate.

53. The method of claim 42 wherein the encapsulated or particulate active compound is a size that is within a range of 30 nm to 1 μm.

54. A method of treating a patient in need thereof with a complement-activating medicinal composition without eliciting a hypersensitivity reaction comprising administering
a) an effective amount of a therapeutic agent wherein said therapeutic agent comprises one or more encapsulated or particulate active compounds wherein said active compounds i) decreases or inhibits the activity of macrophages or monocytes in said patient or ii) diminishes the amount of or eliminates macrophages or monocytes in said patient; and
b) said complement-activating medicinal composition.

55. The method of claim 54 wherein the therapeutic agent is provided as a single dose.

56. The method of claim 54 wherein the therapeutic agent is provided in multiple doses.

57. The method of claim 54 wherein the therapeutic agent is administered intravenously, intramuscularly, intra-arterially, subcutaneously, or orally.

58. The method of claim 54 wherein the therapeutic agents is applied directly to an affected site on the patient.

59. The method of claim 58 wherein the therapeutic agent is coated on a medical device.

60. The method of claim 59 wherein the medical device is a stent.

61. The method of claim 54 wherein the therapeutic agent is administered as an infusion that is completed within one hour.

62. The method of claim 54 wherein the therapeutic agent is administered prior to the administration of the complement-activating medicinal composition.

63. The method of claim 62 wherein the therapeutic agent is administered 2 to 3 days before the administration of the complement-activating medicinal composition.

64. The method of claim 54 wherein the therapeutic agent is administered simultaneously with the administration of the complement-activating medicinal composition.

65. The method of claim 54 wherein at least one of the one or more active compounds is a bisphosphonate.

66. The method of claim 65 wherein the bisphosphonate is selected from the group consisting of alendronate, pamidronate, clondronate, etidronate, and tiludronate.

67. The method of claim 66 wherein the bisphosphonate is alendronate.

68. The method of claim 54 wherein the therapeutic agent is a liposomal bisphosphonate.

69. The method of claim 68 wherein the bisphosphonate is alendronate.

70. The method of claim 54 wherein the complement-activating medicinal composition comprises paclitaxel, Doxil, or a pharmaceutical derivative thereof.

71. The method of claim 54 wherein the complement-activating medicinal composition is a liposomal formulation.

72. The method of claim 54 wherein the encapsulated or particulate active compounds are a size that is within a range of 30 nm to 1 μm.

73. A method for treating or inhibiting a hypersensitivity reaction comprising administering to a patient in need thereof an effective amount of a therapeutic agent wherein said therapeutic agent comprises one or more active compounds that are a size of 30 nm to 1 μm and wherein said active compounds i) decreases or inhibits the activity of macrophages or monocytes in said patient or ii) diminishes the amount of or eliminates macrophages or monocytes in said patient.

74. The method of claim 73 wherein said hypersensitivity reaction is complement activation-related pseudoallergy (CARPA).

75. The method of claim 73 wherein the therapeutic agent is provided as a single dose.

76. The method of claim 73 wherein the therapeutic agent is provided in multiple doses.

77. The method of claim 73 wherein the therapeutic agent is administered intravenously, intramuscularly, intra-arterially, subcutaneously, or orally.

78. The method of claim 73 wherein the therapeutic agent is applied directly to an affected site on the patient.

79. The method of claim 73 wherein at least one of the one or more active compounds is a bisphosphonate.

80. The method of claim 79 wherein the bisphosphonate is selected from the group consisting of alendronate, pamidronate, clondronate, etidronate, and tiludronate.

81. The method of claim 80 wherein the bisphosphonate is alendronate.

82. The method of claim 73 wherein the active compound is encapsulated or particulate.

83. The method of claim 82 wherein the therapeutic agent is a liposomal bisphosphonate.

84. The method of claim 83 wherein the bisphosphonate is alendronate.

85. A method of treating a patient in need thereof with a complement-activating medicinal composition without eliciting a hypersensitivity reaction comprising administering
a) an effective amount of a therapeutic agent wherein said therapeutic agent comprises one or more active compounds one or more active compounds that are a size of 30 nm to 1 μm and wherein said active compounds i) decreases or inhibits the activity of macrophages or monocytes in said patient or ii) diminishes the amount of or eliminates macrophages or monocytes in said patient; and
b) said complement-activating medicinal composition.

86. The method of claim 85 wherein the therapeutic agent is provided as a single dose.

87. The method of claim 85 wherein the therapeutic agent is provided in multiple doses.

88. The method of claim 85 wherein the therapeutic agent is administered intravenously, intramuscularly, intra-arterially, subcutaneously, or orally.

89. The method of claim 85 wherein the therapeutic agents is applied directly to an affected site on the patient.

90. The method of claim 89 wherein the therapeutic agent is coated on a medical device.

91. The method of claim 90 wherein the medical device is a stent.

92. The method of claim 85 wherein the therapeutic agent is administered as an infusion that is completed within one hour.

93. The method of claim 85 wherein the therapeutic agent is administered prior to the administration of the complement-activating medicinal composition.

94. The method of claim 93 wherein the therapeutic agent is administered 2 to 3 days before the administration of the complement-activating medicinal composition.

95. The method of claim 85 wherein the therapeutic agent is administered simultaneously with the administration of the complement-activating medicinal composition.

96. The method of claim 85 wherein at least one of the one or more active compounds is a bisphosphonate.

97. The method of claim 96 wherein the bisphosphonate is selected from the group consisting of alendronate, pamidronate, clondronate, etidronate, and tiludronate.

98. The method of claim 97 wherein the bisphosphonate is alendronate.

99. The method of claim 85 wherein the active compound is encapsulated or particulate.

100. The method of claim 99 wherein the therapeutic agent is a liposomal bisphosphonate.

101. The method of claim 100 wherein the bisphosphonate is alendronate.

102. The method of claim 85 wherein the complement-activating medicinal composition comprises paclitaxel, Doxil, or a pharmaceutical derivative thereof.

103. The method of claim 85 wherein the complement-activating medicinal composition is a liposomal formulation.

104. The method of claim 85 wherein the encapsulated or particulate active compounds are a size that is within a range of 30 nm to 1 μm.
